(19) United States
(12) Patent Application Publication
(10) Pub. No.: US 2008/0248470 A1
(43) Pub. Date: Oct. 9, 2008

(54) GENETIC SCREENING FOR PREDICTING ANTIDEPRESSANT DRUG RESPONSE BASED ON THE MONOAMINE TRANSPORTER GENE POLYMORPHISM COMBINATION

(75) Inventors: DOH KWAN KIM, Seoul (KR); SHINN WON LIM, Seoul (KR); BERNARD J. CARROLL, Carmel, CA (US)

Correspondence Address: MCKEE, VOORHEES & SEASE, P.L.C., 801 GRAND AVENUE, SUITE 3200, DES MOINES, IA, 50309-2721, US

(73) Assignee: SUNGKYUNKWAN UNIVERSITY Foundation for Corporate Collaboration, Suwon-si (KR)

(21) Appl. No.: 11/867,400

(22) Filed: Oct. 4, 2007

(30) Foreign Application Priority Data
Apr. 4, 2007 (KR) 10-2007-0033262
Apr. 4, 2007 (KR) 10-2007-0033263

(51) Int. Cl. C12Q 1/68

(52) U.S. Cl. 435/6

(57) ABSTRACT

The present invention discloses is a method of selecting a drug based on personal genetic information when prescribing an antidepressant for a depressed patient. According to the invention, either a noradrenaline reuptake inhibitor (NRI) antidepressant or a selective serotonin reuptake inhibitor (SSRI) antidepressant can be selected based on the combination of monoamine transporter gene polymorphisms, that is, NET G1287A polymorphism in a norepinephrine transporter (NET) gene, serotonin transporter gene (5-HTT) promoter polymorphism and 5-HTT gene intron 2 polymorphism. Also, based on the norepinephrine transporter gene NET G1287A polymorphism alone, the noradrenaline reuptake inhibitor (NRI) antidepressant can be selected.

### BACKGROUND OF THE INVENTION

1. Field of the Invention

The present invention relates to genetic polymorphism information, which can be used to perform personalized treatment, and more particularly to genetic polymorphism information which is useful for selecting the kind of antidepressant for a depressed patient.

2. Description of the Prior Art

Generally, in drug therapy with antidepressants on depressed patients, responses to the drugs vary depending on the patients. As a result, most commercially available antidepressants show a treatment success rate of only 50-60%. For this reason, there have been attempts to predict treatment responses using the genetic information of individual patients and to provide personalized antidepressants in order to improvement treatment effects. Stober et al. reported that noradrenaline transporter gene NET G1287A polymorphism does not change the function of NET (reference 26) and is not significantly associated with major depression, bipolar disorder, schizophrenia, alcohol dependence or panic disorder (references 27-30).

Meanwhile, it is known that the NET G1287A polymorphism is associated with the cerebrospinal fluid (CSF) concentration of MHPG, a major norepinephrine metabolite (reference 13), and with the response to methylphenidate, a drug with noradrenergic action, in attention deficit/hyperactivity disorder (reference 31). Yoshida et al. previously examined the association between NET polymorphisms and antidepressant responses in Japanese patients (reference 12). They reported that the NET T-182C polymorphism was associated with a superior response to milnacipran, a serotonin-norepinephrine reuptake inhibitor (SNRI), and they also reported that the NET G1287A polymorphism was associated with the onset of response, but not the final clinical improvement.

Pollock et al. examined this polymorphism and the response to nortriptyline (tricyclic antidepressant, noradrenaline reuptake inhibitor) in 23 patients and found no differences. Tsapakis et al. reported the association between 5-HTTLPR and the response to tricyclic antidepressant treatment (reference 32). The report of the present inventors showed that the 5-HTTLPR polymorphism had a significant association with the treatment response to SSRI serotonin reuptake inhibitor antidepressant, (P=0.003), and with the response to nortriptyline, a noradrenaline reuptake inhibitor antidepressant, (P=0.006). However, there are to date no study results suggesting that the kind of drug can be selected based on genetic information about one polymorphism.

### SUMMARY OF THE INVENTION

It is an object of the present invention to provide a marker of norepinephrine transporter (NET) gene polymorphism, which is associated with the treatment effect of an antidepressant.

Another object of the present invention is to provide a haplotype of a norepinephrine transporter (NET) gene, which is associated with the treatment effect of a noradrenaline reuptake inhibitor antidepressant.

Still another object of the present invention is to provide information about the combination of monoamine transporter gene polymorphisms, which is used to selectively screen an antidepressant suitable for an individual patient.

Yet another object of the present invention is to provide information about the combination of monoamine transporter gene polymorphisms, which is suitable for the selection of a noradrenaline reuptake inhibitor antidepressant.

Still yet another object of the present invention is to provide information about the combination of monoamine transporter gene polymorphisms, which is suitable for the selection of a selective serotonin reuptake inhibitor antidepressant.

Another further object of the present invention is to provide a method of detecting said gene polymorphisms from a subject, a method of predicting the treatment effect of an antidepressant based on the detected polymorphisms, and a kit for carrying out the method.

The present invention provides a method in which an antidepressant showing high treatment success rate in an individual patient can be screened through the analysis of genetic information about a norepinephrine transporter (NET) gene, the primary target of a norepinephrine reuptake inhibitor antidepressant, which is typically used in depressed patients, and through the analysis of genetic information about a serotonin transporter gene, the target of a selective serotonin reuptake inhibitor (SSRI) drug. Specifically, the present invention provides a method of screening an antidepressant, having high treatment success rate, using genetic information about NET G1287A in the norepinephrine transporter (NET) gene.

### BRIEF DESCRIPTION OF THE DRAWINGS

FIG. 1 shows a process of administering drugs to patient subjects who participated in the development of the present invention.

FIG. 2 shows changes in depression severity scores measured with the Hamilton depression rating scale during a 6-week period of treatment with a norepinephrine reuptake inhibitor antidepressant.

FIG. 3 shows changes in depression severity scores measured with the Hamilton depression rating scale during a 6-week period of treatment with a selective serotonin reuptake inhibitor antidepressant.

FIG. 4 shows a G1287A polymorphism sequence (Accession X91127; SEQ ID NO: 7) in a NET (norephinephrin transporter) gene.

FIGS. 5a and 5b shows a 5-HTTLPR sequence (Accession X76753 for 16 copies; SEQ ID NO: 8) in the promoter of a serotonin transporter (5-HTT gene), and a VNTR sequence (Accession X76754 for 12 copies; SEQ ID NO: 9) in the intron 2.

### DETAILED DESCRIPTION OF THE INVENTION

Hereinafter, the present invention will be described in detail.

Patients having the GG genotype of the G1287A polymorphism showed a treatment success rate of 58.7% for a selective serotonin reuptake inhibitor (SSRI) drug, but showed a treatment success rate of 83.3% for an NRI drug (58.7% vs. 83.3%, p=0.006, or 3.52 [95% CI (confidence interval), 1.39-8.95]; Table 3). Thus, it is expected that, when an NRI drug prescribed based on the examination of genetic information of NET G1287A is administered to a patient having the GG genotype, the treatment success rate will be increased to 83.35%, which is at least 20% higher than the treatment success rates (50-60%) of prior antidepressants. This result is believed to be clinically significant, considering that depressed patients with the GG genotype of the NET polymorphism in Korea is 56% of the population. The clinical application of the present invention enables the kind of antidepressant having high treatment effect to be previously screened even by the examination of only one genotype from patient's blood, thus achieving personalized therapy.

In a previous study (reference 6), the present inventors found that the alleles of a 5-HTT gene, a serotonin transporter gene, were associated with variation in antidepressant responses to SSRIs. In the present invention, the approach conducted in said previous study was extended to the allelic variation of the noradrenaline (norepinephrine) transporter (NET). The allele-containing NET of the present invention is known to be the target of the norepinephrine reuptake inhibitor (NRI) among antidepressant drugs. Also, the present invention included the dopamine transporter (DAT) polymorphism (reference 13).

On the basis of said previous study, the present inventors selected, as candidate gene variants for the prediction of antidepressant responses, the 5-HTTLPR (5-HTT gene-linked promoter region; NCBI Genbank site (http://www.ncbi.nlm.nih.gov/); accession number AF117826; positions 25,584,988-25,585,338 of chromosome 17) and intron 2 VNTR (variable number of tandem repeat; positions 25,570,101-25,570,300 of chromosome 17) of the 5-HTT gene (references 1 and 5), the G1287A polymorphism (NCBI SNP address: rs5569, Thr429Thr) in exon 9, the C296T polymorphism (rs1805065, Thr99Ile) in exon 2 and the G1432A polymorphism (rs1805067, Gly478Ser) in exon 10 of the NET gene (references 14 and 15), and the 3′-untranslated region VNTR of the DAT gene (reference 13). The C296T and G1432A polymorphisms of NET and the 3′-untranslated region VNTR of DAT were excluded from the analysis, because minor alleles of these polymorphisms were relatively uncommon (less than 5% of the population). As used herein, the term “G1287A polymorphism of NET” refers to a G-to-A substitution at position 1287 from the translation start site of NET and is a synonymous variant in which threonine amino acid at position 229 is not changed due to base sequence variation.

Information about said 5-HTT gene and NET gene is as follows.

Serotonin Transporter (5-HTT Gene)

Official Symbol: SLC6A4 and Name: solute carrier family 6 (neurotransmitter transporter, serotonin), member 4 [Homo sapiens].

Other Aliases: 5-HTT, 5HTT, HTT, OCD1, SERT, hSERT.

Other Designations: 5-hydroxytryptamine transporter; 5HT transporter; Na+/Cl-dependent serotonin transporter; serotonin transporter; sodium-dependent serotonin transporter, solute carrier family 6 member 4.

Chromosome: 17; Location: 17q11.1-q12.

MIM: 182138.

GeneID: 6532.

Norephinephrin Transporter (NET Gene)

Official Symbol: SLC6A2 and Name: solute carrier family 6 (neurotransmitter transporter, noradrenalin), member 2 [Homo sapiens].

Other Aliases: NAT1, NET, NET1, SLC6A5.

Other Designations: noradrenaline transporter; norepinephrine transporter; solute carrier family 6 (neurotransmitter transporter, norepinephrine), member 5; solute carrier family 6 member 2.

Chromosome: 16; Location: 16q12.2.

MIM: 163970.

GeneID: 6530.

As primary hypotheses, the present inventors predicted the associations between NRI efficacy and NET polymorphisms and between SSRI efficacy and 5-HTT polymorphisms. Also, the present inventors compared the response rates to NRI and SSRIs by genotype. In additional secondary analyses, the present inventors examined combinations of polymorphisms and the response to NRI or SSRIs. The present inventors selected nortriptyline as NRI, and fluoxetine and sertraline as SSRI (selective serotonin reuptake inhibitor). These drugs are the agents most commonly used to treat late-life depression in Korea. In addition to treatment responses, the present inventors also monitored adverse events to antidepressants by the UKU side effect rating scale (score scale for evaluating side effects in patients administered with psychotropic drugs).

In the present invention, experiments were conducted on patients other than the patient group of a prior report (reference 6). The patient subjects who participated in the experiments of the present invention were all 18-years-old and were enrolled in the Clinical Trials Program of the Samsung Medical Center Geropsychiatry and Affective Disorder Clinics (Seoul Korea). The affective disorder section of the Samsung Psychiatric Evaluation Schedule (SPES) uses the Korean version of the Structured Clinical Interview for DSM-IV Axis-I disorders (SCID-IV; 4^{th} edition, reference 17).

In the present invention, at least one family member who is living with the patient was interviewed to supplement the patient's report of symptoms, behaviors, level of functioning, duration of episode, and recent treatments. The patient subjects who participated in the experiments of the present invention all fulfilled DSM-IV criteria for major depressive episode, the Korean version of the Structured Clinical Interview for DSM-IV Axis-I disorders (4^{th} edition). Diagnoses were confirmed by a board certified psychiatrist on the basis of the SPES, case review notes, and other relevant data. A minimum baseline 17-item Hamilton depression scale (HAM-D) score of 15 was required.

Patient subjects, who received other psychotropic medications within 2 weeks of the study or fluoxetine within 4 weeks, were excluded. In the present invention, potential subjects were excluded for pregnancy, significant medical conditions, abnormal laboratory baseline values, unstable psychiatric features (e.g., suicidality), history of alcohol or drug dependence, seizures, head trauma with loss of consciousness, neurologic illness, or concomitant Axis I psychiatric disorder. The protocol was approved by the ethics review board of Samsung Medical Center, Seoul, Korea. Signed informed consent was obtained from all participants.

A total of 241 patients were subjected to the experiment of the present invention during a period from March, 1998 to February, 2003. The patients were assigned to monotherapy with the NRI antidepressant (nortriptyline) or the SSRI antidepressant (fluoxetine or sertraline). Clinician's choice of drug was driven by anticipated side effects of nortriptyline in at-risk individuals rather than by the symptomatic characteristics of the patients. Factors that determined use of fluoxetine or sertraline in preference to nortriptyline were frailty, osteoporosis, a history of falls, and known cardiovascular disease. 105 patients received nortriptyline, and 136 patients received SSRIs (fluoxetine (n=51), sertraline (n=85)). Dose titration was completed within 2 weeks. Doses were titrated into the usual clinical range based on initial tolerability and side effects. The final daily median (interquartile range, range) dosages were 55 (47.5-70, 35-100) mg/day of nortriptyline, 30 (20-40, 20-50) mg/day of fluoxetine, and 75 (75-100, 50-100) mg/day of sertraline. These are typical clinical doses for Asian populations, and they result in comparable plasma drug levels as do higher drug dosages in white populations (reference 18). Trough serum samples to measure NRI and SSRI levels were drawn at the end of week 4. 1-2 mg of Lorazepam could be prescribed at bedtime for insomnia. In the present invention, the patients were seen by a psychiatrist, who monitored their adverse events by the UKU side effect rating scale (reference 16) at week 0, 0.5, 1, 2, 4, and 6. The 17-item HAM-D was administered by a single trained rater every 2 weeks. The rater and genotype were blinded to the hypotheses of the study and drug assignment. To maintain the blindness, a trained research coordinator managed the data and schedules. HAM-D and genotype data were not disclosed to the psychiatrist, and the rater was blinded to the genotype data.

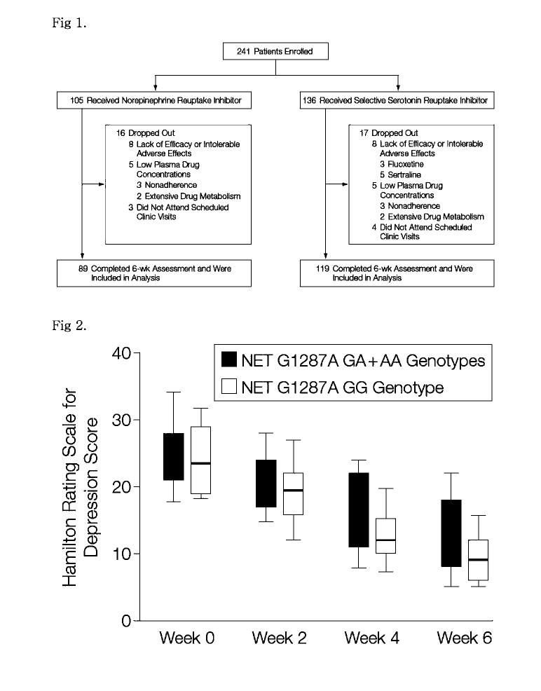

In the present invention, response to drugs was defined as a 50% or greater decrease in the HAM-D score at 6 weeks. Remission was defined as a HAM-D of less than 8 at 6 weeks (reference 19). FIG. 1 shows the flow of patients through the experiment of the present invention.

In the present invention, 208 patients (86% of the original participants) completed the six-week treatment trial. Sixteen patients receiving NRI treatment and 17 patients receiving SSRI treatment dropped out. Eight patients receiving nortriptyline, three patients receiving fluoxetine and five patients receiving sertraline discontinued treatment based on lack of efficacy or intolerable side effects. Ten patients were excluded, either because their plasma drug concentrations were undetectable, consistent with noncompliance (N=6) or were in the range consistent with extensive drug metabolism (<200 ng/ml norfluoxetine-fluoxetine and <30 ng/ml sertraline, N=4) (reference 20). Seven patients failed to attend for scheduled clinic visits. The dropouts did not differ significantly by clinical characteristics from the completers who received either SSRI or NRI (data not shown). These 33 dropouts were excluded in data analyses.

The results of the present invention indicate that: antidepressant response to NRI is principally associated with the NET G1287A polymorphism; response to NRI is also secondarily related to the 5-HTTLPR polymorphism; response to SSRI is associated with the 5-HTT intron 2 and 5-HTTLPR polymorphisms; and response to SSRI is not related to the NET G1287A polymorphism. Thus, the primary hypotheses of the present inventors were confirmed and extended.

The present inventors observed significant associations of NRI efficacy with NET G1287A polymorphism and of SSRI efficacy with 5-HTT polymorphisms. These findings support the concept that pharmacologically selective antidepressants act primarily through homologous neurotransmitter mechanisms. Thus, these findings are consistent with observations that selective depletion of monoamines leads to relapse in patients who have responded to the homologous monoamine transporter inhibitors (references 24 and 25). These results of the present invention support the primary hypothesis that responses to NRI and SSRI are significantly associated with their respective transporter polymorphisms. The results of the present invention suggest that the NET G1287A polymorphism plays a major role in the NRI antidepressant response. The 5-HTTLPR polymorphism was significantly associated with the responses to both NRI and SSRI. This suggests that the 5-HTTLPR polymorphism is generally associated with the response to multiple interventions for depression, including drugs of several classes, placebo, sleep deprivation and light therapy. It was reported in a white population that patients with the long allele (especially ll genotype) of 5-HTTLPR are more responsive to placebo, sleep deprivation and light therapy as well as more responsive to drug than those with the short allele (references 4 and 36).

The present inventors replicated our previous finding that a variable repeat sequence of 5-HTTLPR is associated with response to SSRI drugs in depressed patients (p=0.003; Table 3), even though the favorable allelic variant for response again is at odds with studies in white patients (references 1-5). The reason for this ethnic difference remains unclear. However, the data may suggest several speculations.

First, allelic frequencies for 5-HTTLPR in Korean/Japanese populations differ greatly from white populations. The allele frequency of l variant 5-HTTLPR in Korean/Japanese populations is about 25% (references 1-3 and 39) compared with about 55% in white populations (references 6, 7, 37 and 38). It is assumed from studies of white patients that the l variant 5-HTTLPR is the favorable allele for response to SSRIs, it is expected that the rate of response of Korean/Japanese patients to those agents will be low. However, a consistent finding is that 60-70% of depressed patients respond to SSRI drugs in multi-center clinical trials regardless of ethnic group (references 40-43). The second speculation is that 5-HTTLPR is linked with unknown functional variants. 5-HTTLPR may be an associated marker in linkage disequilibrium with a functional site, rather than a functional polymorphism itself. The authentic functional sequence variants may be in strong linkage disequilibrium with l allele in the white population and also in linkage disequilibrium with s allele in Koreans. For similar speculation on the 2^{nd} intronic VNTR of 5-HTT gene, there may be a functional site closely linked with the 2^{nd} intronic VNTR in the Korean population, not in the white, that is associated with SSRI response. Different ethnic populations may have other polymorphisms in linkage disequilibrium with 5-HTT polymorphisms. Moreover, it may not be possible to explain the ethnic difference by analyzing only these two polymorphisms. Thus, although studies to date have focused on these two polymorphisms of the 5-HTT gene, the ethnic variation observed in this study suggests that these alleles are only indirectly responsible for the observed interactions with treatment response. New approaches that include whole gene sequencing or entire SNP analyses are needed to identify the responsible loci.

In this regard, the present inventors note that the current SNP database from the NCBI web site shows 111 SNPs in the 5-HTT gene (http://www.ncbi.nlm.nih.gov/), and all of these should be considered in the search for functional polymorphisms. The finding of the present invention illustrates the importance of comparing ethnic groups to confirm candidate pharmacogenetic markers.

As to the prediction of differential drug response, the preliminary data analysis of the present inventors suggests that patients carrying the GG polymorphism of NET G1287A will have a statistically significantly superior rate of response to NRI treatment than to SSRI treatment (83.3% NRI vs. 58.7% SSRI; p=0.0006; OR=3.52; 95% confidence interval, 1.39-8.95). As this genetic subgroup comprised 56% of the population (117 of 208 cases), this result may prove to have salience for clinical practice. This preliminary finding needs to be tested in studies specifically designed to examine differential response to drug class by genotype.

At least some of the individual variation in antidepressant treatment outcome has a genetic basis (reference 44). Although the functional influence of these transporter polymorphisms is not fully understood, they are related to the transcription of individual genes. The l and s variants of the promoter polymorphism have functional differences in modulating transcription of the 5-HTT gene as well as subsequent 5-HTT availability (reference 45). These allele-specific functional differences have been confirmed in human tissues including the brain (references 46 and 47). Thus, the 5-HTT polymorphisms might influence response to treatment by modulating transcription of 5-HTT, a direct target of SSRIs.

Exploratory post hoc analyses conducted by the present inventors point to the predictive potential of combinations of polymorphisms. Two significant polymorphisms, NET G1287A and 5-HTT promoter polymorphisms, contributed to the prediction of NRI response. For instance, in patients camming the A allele of the NET G1287A polymorphism, the rate of response to nortriptyline was 43% (20/47) (Table 4). After stratifying by the 5-HTT promoter polymorphism, however, the rates of response to nortriptyline were 63% (15/24) in those with the ‘NET A+promoter ss’ combination, and only 22% (5/23) in those with the ‘NET A+promoter l’ combination. For SSRIs, two 5-HTT polymorphisms contributed to the prediction of response, with no contribution from NET polymorphism. As the number of favorable genotypes (ss in 5-HTTLPR, and ll in Intron 2) increased, the response rate to SSRIs increased (77.4% for 2 favorable genotypes, 25.0% for 1 favorable genotype, and 0% for no favorable genotype). Notwithstanding the small size of the subgroup samples for these secondary analyses, these differential response rates by polymorphism combination are statistically significant and they are potentially clinically meaningful. It is also interesting that the effect of the 5-HTT intron 2 polymorphism varies depending on drugs. That is, even within a single gene, the association between this polymorphism and outcome is drug-class specific. Likewise, the effect of the NET G1287A polymorphism is drug-class specific.

In the present invention, the patients were mostly elderly (77% over age 50), and most (60%) had late onset illnesses with few previous depressive episodes. Eighty-eight percent of all cases were in their first or second lifetime episode of depression. The present inventors adopted strict criteria for a previous major depressive episode, excluding minor depression or dysthymia. It is unclear whether late-life depression has distinctivegenetic contributions. It is generally accepted that familial risk in affective disorder is reduced after 50 years, and that patients with late-onset depression are less likely to have psychiatric co-morbidity and more likely to have medical co-morbidity. However, previous studies have demonstrated that depression symptoms in older adults might be more heritable than previously thought (reference 50), and that early onset and late onset groups do not differ from each other in genotype frequency distribution of the two 5-HTT gene polymorphisms (references 51 and 52). Likewise, it is unknown whether antidepressant response and pharmacogenetic effects are affected by age or by age of onset (references 52 and 53).

The present inventors found no differences of genotype distributions between early onset (onset age 59 or younger, n=126) and late onset (onset age 60 or older, n=82) patients (p=0.25, 0.82 and 0.97 by chi-square (χ²) tests for 5-HTTLPR, 5-HTT intron 2, and NET G1287A, respectively). The result was similar when the present inventors compared genotype distributions between mid-life (age 59 or younger, n=89) and late-life (age 60 or older, n=19) patients (p=0.30, 0.35 and 0.44 by chi-square tests for 5-HTTLPR, 5-HTT intron 2, and NET G1287A, respectively). As for treatment response, some previous pharmacogenetic studies reported similar results with elderly and younger patients when controlling on ethnicity and drug (references 4, 8 and 54). The study of the present invention demonstrates that the responses to antidepressants with different targets have significant associations with homologous monoamine transporter gene polymorphisms. The data of the present invention confirm a relationship between SSRI response and 5-HIT polymorphisms, and establish an association between NRI response and the NET G1287A polymorphism. The present inventors also found that the 5-HTTLPR s/l variation plays a role in the treatment of depression with both NRI and SSRI agents.

Hereinafter, the present invention will be described in further detail with reference to examples. It is to be understood, however, that these examples are illustrative only, and the scope of the present invention is not limited thereto. All the literature cited in the present specification is incorporated herein by reference.

### EXAMPLES

### Example 1

### Genotype Analysis

Genomic DNA was extracted from whole blood using a Wizard Genomic Purification kit (Promega, Madison, Wis., USA). The present inventors analyzed the genotype of the 5-HTT promoter s/l polymorphism (5-HTTLPR), the genotype of the 5-HTT intron 2 s/l polymorphism, and the NET G1287A polymorphism in exon 9. For this purpose, the G1287A polymorphism in exon 9 was amplified by PCR with primers of SEQ ID NO: 1 and SEQ ID NO: 2 and digested by restriction enzyme Sau96I. 8F (5′-TCCAGGGAGACCCTAATTCC) (SEQ ID NO: 1) and 8R (5′-TTGACTTTATTGAAATGCGGC) (SEQ ID NO: 2). The PCR was carried out in a total volume of 25 μl containing 40 ng genomic DNA, 0.2 mM dNTP, 10 pmol of primers, 10 mM Tris-HCl (Ph 8.3), 50 mM KCl, 3.5 mM MgCl₂, 0.1% Triton-X100, and 0.5 U Taq polymerase. The PCR reaction was performed in the following conditions: pre-denaturation at 94° C. for 5 min, and then 40 cycles at 94° C. for 30 sec, 57° C. for 45 sec, and 72° C. for 45 sec in series, followed by post-elongation at 72° C. for 10 min. The PCR products were digested at 37° C. for 1 hr with restriction enzyme Sau96I and detected on 12% polyacrylamide gel. Depending on the absence or presence of the polymorphic Sau96I site, either a 1287A (113+97+31 bp) or 1287G (113+76+31+21 bp) fragment was produced. The VNTR polymorphism in the intron 2 region and the 5-HTTLPR (5-HTT linked polymorphic region) element in the promoter region were also detected through PCR amplification.

For the analysis of VNTR in intron 2 of the serotonin transporter gene, the VNTR region in intron 2 of the serotonin transporter gene containing 17 repeat sequences was amplified by PCR with primers 8224 (5′-GTCAGTATCACAGGCTGCGAG) (SEQ ID NO: 3) and 8223 (5′-TGTTCCTAGTCTTACGCCAGTG) (SEQ ID NO: 4). The PCR was carried out in a mixture containing 20 ng genomic DNA, 50 mM KCl, 10 mM Tris.HCl (pH 9.0 at 25° C.), 0.1% Triton-X100, 1 mM MgCl₂, 0.2 mM dNTP, 1 μl Taq polymerase, and 1 μM of each of sense- and antisense-primers. The PCR reaction was performed in the following conditions: pre-denaturation at 94° C. for 3 min, and then 25 cycles of denaturation at 94° C. for 30 sec, annealing at 60° C. for 45 sec and elongation at 72° C. for 45 sec, followed by post-elongation at 72° C. for 8 min. The PCR amplification products were electrophoresed on 3% agarose gel to confirm bands having 9 and 10 copies (s allele) and 12 copies (1 allele), compared to a pUC 18 Hae III digestion marker (Sigma). The 9- and 10-copy VNTR were designated “s allele of 5-HTT intron 2”, and the 12-copy VNTR was designated “l allele”.

For the analysis of the 5-HTT promoter s/l polymorphism (5-HTTLPR) of the serotonin transporter gene, PCR reaction was performed using primer stpr5 (5′-GGCGTTGCCGCTCTGAATTGC (SEQ ID NO: 5); corresponding to positions −1,416 to −1,397 of the nucleotide) and primer stpr3 (5′-GAGGGACTGAGCTGGACAACCCAC (SEQ ID NO: 6); corresponding to positions −910 to −889 of the nucleotide). The PCR amplification was performed in a mixture containing 0.1 mM dNTP, 0.15 μM sense and antisense primers, 150 ng genomic DNA, 2 mM Tris.HCl (pH 7.5 at 25° C.), 10 mM KCl, 0.1 mM dithiothreitol (DTT), 0.01 mM EDTA, 0.05% Tween20 (v/v), 0.05% Nonidet P40 (v/v), 5% glycerol, and 1.3μ expand high fidelity PCR system enzyme mix (Boehringer Mannhein, Mannhein, Germany), in the following conditions: pre-denaturation at 95° C. for 4 min, 10 cycles of denaturation at 95° C. for 30 sec, annealing at 65° C. for 30 sec and elongation at 72° C. for 45 sec, and then 20 cycles of denaturation at 95° C. for 30 sec, annealing at 65° C. for 30 sec and elongation at 72° C. for 4 min and 5 sec, followed by post-elongation at 72° C. for 7 min. The amplified products were electrophoresed on 2% agarose gel to confirm bands having 14 copies (s allele), 16, 18, 20 and 22 copies (defined as l allele for more than 16 copies), compared to a 100-bp ladder marker (reference 6). The 14-copy VNTR of 5-HTTLPR was designated “s allele”, and the 16-, 18-, 20- and 22-copy VNTRs were designated “l alleles”.

### Example 2

### Detection of Plasma Drug Levels

Plasma levels of nortriptyline, fluoxetine and sertraline were quantified according to conventional methods with liquid chromatography tandem mass spectrometry (HPLC-MS/MS) (references 20-22).

### Example 3

### Statistical Analysis

Means and standard deviations (SDs) and ranges of continuous variables, and proportions of categorical variables, are presented as descriptive statistics. The present inventors employed the Mann-Whitney U test on continuous variables, as these were not normally distributed, and the chi-square (χ²) test on categorical variables. Hardy-Weinberg equilibrium was tested by the chi-square test. Power analyses were performed to examine if the number of patients was sufficient to produce a statistically significant result, given a true difference. Comparisons of the genotype frequencies and allele frequencies between the antidepressant responders and non-responders were performed using Fisher's exact test. Multiple logistic regression model entering all three genes was used to evaluate the influence of each gene on the response to the medication adjusting for other genes. Bonferroni's correction was applied to multiple testing. Results were considered significant at P<0.05 after this correction. P-values from Bonferroni's correction were stated with the corrected values. Limited exploratory, post hoc analyses were conducted with Fisher's exact test using permutation method for multiple testing to examine response rates in relation to genotype combinations. The same method was used to compare differential response to NRI or SSRI by genotype. Measures of linkage disequilibrium (LD) were calculated using the Gold program. All the statistical analyses were performed using SAS software version 9.13 (SAS Institute Inc, Cary, N.C.).

Results

There were no major differences by gender, number of episodes, age of onset, or HAM-D scores before or after treatment between the NRI-treated and SSRI-treated groups (Table 1). Patients treated with SSRIs (mean [SD] age: 59.9 [12.6] years old) were older than those given nortriptyline (mean [SD] age: 55.8 [12.4] years old). The mean age of onset of major depressive disorder was in the early to mid-50s (Table 1). Rate of response to antidepressants was 124 of 208 patients (60%) who completed the 6-week treatment trial, 62% (55/89) for NRI and 58% (69/119) for SSRIs (p=0.58). Rate of remission did not differ by drug class (29% (26/89) for NRI and 29% (34/119) for SSRI). The overall distribution of response, remission, and nonresponse did not differ by drug class (p=0.84).

TABLE 1
Characteristics of Study Patients

| | | | | |
|---|---|---|---|---|
| Characteris- | | | | |
| tics | Total | Responder | Non-responder | P |

NRI-treated group

| | | | | |
|---|---|---|---|---|
| Response | 55/89 (61.8) | | | |
| Rate (%)* | | | | |
| Gender* | | | | 0.89 |
| Male | 19 | 12 | 7 | |
| Female | 70 | 43 | 27 | |
| Age, | 55.80 (12.40) | 54.78 (11.85) | 57.44 (13.25) | 0.33 |
| years †‡ | [22-76] | [26-76] | [22-74] | |
| Mean ± SD | | | | |
| (Range) | | | | |
| Number of | 1.72 (1.87) | 1.58 (1.40) | 1.94 (2.46) | 0.38 |
| Episodes^{†} | [1-15] | [1-10] | [1-15] | |
| Mean ± SD | | | | |
| (Range) | | | | |
| Onset Age, | 51.01 (13.69) | 50.80 (13.37) | 51.35 (14.39) | 0.85 |
| years †‡ | [22-76] | [23-76] | [22-73] | |
| Mean ± SD | | | | |
| (Range) | | | | |
| HAM-D | 24.88 (5.82) | 24.67 (6.07) | 25.21 (5.45) | 0.99§ |
| Baseline^{†} | [15-24] | [15-42] | [17-41] | |
| Mean ± SD | | | | |
| (Range) | | | | |
| HAM-D | | | | |
| After^{†} | | | | |
| Mean ± SD | | | | |
| (Range) | | | | |

SSRI-treated group

| | | | | |
|---|---|---|---|---|
| Response | 69/119 (58.0) | | | |
| Rate (%)* | | | | |
| Gender* | | | | 0.38 |
| Male | 33 | 17 | 16 | |
| Female | 86 | 52 | 34 | |
| Age, | 59.91 (12.61) | 58.12 (13.86) | 62.38 (10.28) | 0.07 |
| years †‡ | [22-85] | [22-81] | [38-85] | |
| Mean ± SD | | | | |
| (Range) | | | | |
| Number of | 1.90 (2.41) | 1.68 (0.99) | 2.20 (3.53) | 0.25 |
| Episodes ^{†} | [1-25] | [1-6] | [1-25] | |
| Mean ± SD | | | | |
| (Range) | | | | |
| Onset Age, | 54.87 (14.08) | 52.62 (15.67) | 57.96 (10.94) | 0.03 |
| years †‡ | [19-81] | [19-81] | [35-80] | |
| Mean ± SD | | | | |
| (Range) | | | | |
| HAM-D | 23.52 (5.11) | 23.55 (5.63) | 23.48 (4.33) | _.99§ |
| Baseline^{†} | [16-40] | [16-40] | [16-36] | |
| Mean ± SD | | | | |
| (Range) | | | | |
| HAM-D | 11.66 (5.94) | 7.75 (3.23) | 17.04 (4.40) | .01§ |
| After^{†} | [1-31] | [1-15] | [10-31] | |
| Mean ± SD | | | | |
| (Range) | | | | |

*χ² test;
† t-test;
‡ P < 0.05 for comparison between the SSRI-treated group and the NRI-treated group;
§ Corrected value by Bonferroni's correction for multiple testing.

Genotype distribution for each monoamine transporter polymorphism was not significantly different between the NRI group and SSRI group (Table 2), and was not significantly different within the SSRI group (fluoxetine-treated patients vs. sertraline-treated patients). The choice of drug in the SSRI group (fluoxetine vs. sertraline) had no effect on drug responsiveness (p=0.56). Genotypes were unrelated to dropout status or summed total score of the UKU side effect rating scale in either the NRI- or SSRI-treated groups. There were no statistical differences in genotype distributions between early-onset depression (EOD, lifetime onset ≦age 59) and late-onset depression (LOD, lifetime onset ≧60) (p=0.25, 0.82, and 0.97 for 5-HTTLPR, 5-HTT intron 2, and NET G1287A, respectively). Comparisons between younger (≦age 59) and older patients were similarly insignificant (p=0.30, 0.35, and 0.44, respectively). Plasma levels (mean ±SD) of nortriptyline (116±21 vs. 123±23 ng/ml), fluoxetine-norfluoxetine (789±236 vs. 802±206 ng/ml) and sertraline (75±29 vs. 72±28 ng/ml) were not significantly different between responders and non-responders. The responders did not differ significantly by lorazepam dosages from the nonresponders who received either SSRI or NRI (p=0.71 and 0.46, respectively).

TABLE 2
Genotype distributions of monoamine transporter gene polymorphisms
Number (%) of patients

| | All Study | | | |
|---|---|---|---|---|
| | Subjects | NR1 group | SSRI group | P |
| | (N = 208) | (N = 89) | (N = 119) | Value* |
| NET G1287A | | | | |
| in exon 9 | | | | |
| GG | 117 (56.3) | 42 (47.2) | 75 (63.0) | |
| GA | 78 (37.5) | 41 (46.1) | 37 (31.1) | 0.21 |
| AA | 13 (6.3) | 6 (6.7) | 7 (5.9) | |
| 5-HTT VNTR | | | | |
| in promoter | | | | |
| ss | 120 (57.7) | 50 (56.2) | 70 (58.8) | |
| sl | 71 (34.1) | 29 (32.6) | 42 (35.3) | 0.99 |
| ll | 17 (8.2) | 10 (11.2) | 7 (5.9) | |
| 5-HTT VNTR | | | | |
| in intron 2 | | | | |
| ll | 174 (83.7) | 77 (86.5) | 97 (81.5) | |
| ls | 33 (15.9) | 12 (13.5) | 21 (17.6) | 0.99 |
| ss | 1 (0.5) | 0 (0) | 1 (0.8) | |

Abbreviations: 5-HTT, serotonin transporter;
NET, norepinephrine transporter;
NRI, noradrenergic reuptake inhibitor,
SSRI, selective serotonin reuptake inhibitor;
and VNTR, variable number of tandem repeats.
*Comparisons of the genotype frequencies between the NRI group and the SSRI group were performed using χ² test followed by Bonferroni's correction for multiple testing.

### NRI Response and Monoamine Transporter Gene Polymorphisms

The genotype frequencies in the NRI-treated group for the 5-HTTLPR, 5-HTT intron 2 and NET G1287A polymorphisms were in Hardy-Weinberg equilibrium (p=0.23, 1.00, and 0.34, respectively). For the power analysis in the NRI-treated group, the response rate of patients with the GG genotype was predicted to be 80%, and the response rate of patients with the GA+AA genotypes was predicted to be 50%. With 42 patients with GG genotype and 47 patients with GA+AA genotypes, the power to detect this difference of 30% was 80% under the significance level of 5%. Response to NRI was strongly associated with the NET G1287A polymorphism (p=0.001 by multiple logistic regression, OR=7.54, 95% CI (2.53-22.49)). A response rate of 83.3% (35/42) was associated with the GG genotype, significantly greater than the response rate of 42.6% (20/47) in the GA+AA genotypes (p=0.01, Table 3). The GA and AA genotypes were combined for this analysis, because the 1287AA genotype was found in only 6 patients. The reduction in HAM-D score after 6 weeks of NRI treatment was greater in the GG genotype than in GA+AA genotypes (p=0.006, FIG. 2). At 6 weeks, a significant difference in the Hamilton rating scale for depression scores was found between the GG group and the GA+AA group. In FIG. 2, the change in depression score for the G genotype was 0.14 (95% CI, 0.19-0.11), and the change in depression score for the GA+AA genotypes was 0.11 (95% CI, 0.15-0.6) (p=0.006 by Mann-Whitney U test). In FIG. 2, each box displays the median, 75^{th} percentile, and 25^{th} percentile values, and horizontal bars indicate the highest and lowest observed values. NET indicates the norepinephrine transporter. The NRI response was also associated with the 5-HTT promoter region polymorphism (p=0.01 by multiple logistic regression; OR=3.73; 95% CI, 1.32-10.53). The favorable allele for the NRI response was s allele of 5-HTT promoter polymorphism (p=0.003, Table 3). A response rate of 76% (38/50) to the NRI was associated with the ss genotype of the 5-HTT promoter polymorphism. In comparison, the response rates to the NRI were 48% (14/29) in those with the sl genotype, and 30% (3/10) in patients who carried the ll genotype. The frequency of the 5-HTT promoter ss genotype was significantly higher in the NRI responders than in the non-responders (p=0.006). The l/s polymorphism of 5-HTT intron2 region had no relation with the NRI response (p=0.13 by multiple logistic regression; OR=3.34; 95% CI, 0.70-15.91).

TABLE 3
Genotype and allele distributions of monoamine transporter gene polymorphisms in
responders and non-responders to antidepressants

| | | | | | |
|---|---|---|---|---|---|
| | | Non- | | | |
| Response rate | Responders | responders | | OR (97% CI) | |
| (%) | (%) | (%) | P value * | † | P value † |

NRI-treated group

| | | | | | | |
|---|---|---|---|---|---|---|
| NET | | | | | | |
| G1287A | | | | | | |
| in exon | | | | | | |
| 9 | | | | | | |
| GG | 35/42(83.3) | 35(63.6) | 7(20.6) | | | |
| GA | 16/41(39.0) | 16(29.1) | 25(73.5) | 0.1 | 7.54(2.53- | .001 |
| | | | | | 22.49) | |
| AA | 4/6(66.7) | 4(7.3) | 2(5.9) | | | |
| G | | 0.78 | 0.57 | 0.1 | 3.48(1.67- | .001 |
| | | | | | 7.30) | |
| A | | 0.22 | 0.43 | | | |
| 5-HTT | | | | | | |
| VNTh | | | | | | |
| in | | | | | | |
| promoter | | | | | | |
| ss | 38/50(76.0) | 38(69.1) | 12(35.3) | .000 | 3.73(1.32 | .01 |
| | | | | | 10.53) | |
| sl | 14/29(48.3) | 14(25.5) | 15(44.1) | | | |
| ll | 3/10(30.0) | 3(5.4) | 7(20.6) | | | |
| S | | 0.82 | 0.57 | .003 | 4.85(2.29- | .001 |
| | | | | | 10.27) | |
| L | | 0.18 | 0.43 | | | |

SSRI-treated group

| | | | | | | |
|---|---|---|---|---|---|---|
| NET | | | | | | |
| G1287A | | | | | | |
| in exon | | | | | | |
| 9 | | | | | | |
| GG | 44-75(58.7) | 44(63.8) | 31(62.0) | | | |
| GA | 21/37(56.8) | 21(30.4) | 16(32.0) | >.99 | 0.84(0.34- | .71 |
| | | | | | 2.09) | |
| AA | 4/7(57.1) | 4(5.8) | 3(6.0) | >.99 | 1.54(0.74- | .25 |
| | | | | | 3.20) | |
| G | | 0.79 | 0.78 | | | |
| A | | 0.21 | 0.22 | | | |
| 5-HTT | | | | | | |
| VNTR | | | | | | |
| in | | | | | | |
| promoter | | | | | | |
| ss | 50/70(71.4) | 50(72.5) | 20(40.0) | .0.03 | 3.34(1.41- | .006 |
| | | | | | 7.91) | |
| sl | 17/42(40.5) | 17(24.6) | 25(50.0) | | | |
| ll | 2/7(28.6) | 2(2.9) | 5(10.0) | | | |
| S | | 0.85 | 0.65 | .003 | 2.28(1.17- | .02 |
| | | | | | 4.47) | |
| L | | 0.15 | 0.35 | | | |

SSRI Response and Monoamine Transporter Gene Polymorphisms

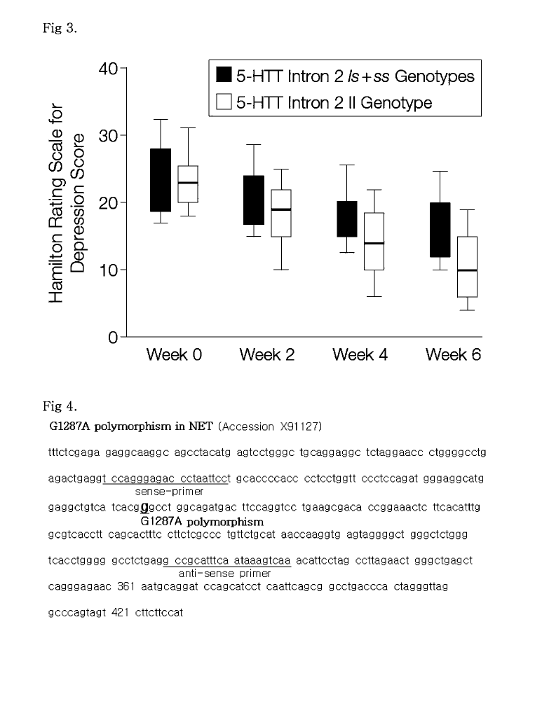

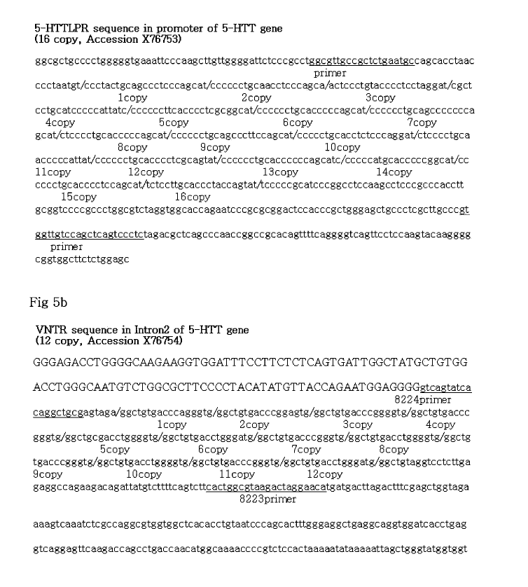

Within the SSRI-treated group, the genotype frequencies for the 5-HTT promoter, 5-HTT intron 2, and NET G1287A polymorphisms were in Hardy-Weinberg equilibrium (p=0.83, 1.00, and 0.40, respectively). For the power analysis, the response rate of the patients with ll genotype in 5-HTT intron 2 was assumed to be 70%, compared with 30% in the patients with ls+ss genotypes. With 97 patients with ll and 22 patients with is ls+ss, the power of detecting this difference of 40% was 90% under the significance level of 5%. Table 3 shows the genotype distribution and allele frequencies in the SSRI responders and non-responders. Response to SSRI was significantly associated with the 5-HTT intron2 polymorphism and the 5-HTT promoter polymorphism. Patients with the ll genotype of 5-HTT intron2 had a 69% (67/97) rate of response to SSRIs, compared with only 9% (2/22) for the other 2 genotypes combined (p=0.01). The 2^{nd} intronic polymorphism of the 5-HTT gene showed the strongest association with SSRI response among the monoamine transporter gene polymorphisms (p<0.001 by multiple logistic regression, OR=20.11; 95% CI, 4.27-94.74). FIG. 3 shows the difference in HAM-D score reduction after 6 weeks of SSRI medication between the two 5-HTT intron 2 genotype groups (ll genotype vs. ls+ss genotypes, p<0.001). As shown in FIG. 3, significant difference was found in Hamilton depression rating score changes between the ll group and the ls+ss group at week 6. HAM-D score changes at week 6 were −12 [95% CI, −16.5 to −9] for ll genotype, and −6.5 [95% CI, −8.75 to −5] for ls+ss genotypes (p<0.001 by Mann-Whitney U test). In FIG. 3, each box displays the median, 75^{th} percentile, and 25^{th} percentile values; horizontal bars indicate the highest and lowest observed value. Also, 5-HTT indicates the serotonin transporter. Moreover, response to SSRI was also significantly associated with polymorphism of the 5-HTT promoter region. Patients with the ss genotype at this site had a 71% (50/70) rate of response to SSRI, compared with rates of 40% (17/42) with the ls genotype and 29% (2/7) with the ll genotype (ss genotype vs. sl+ll genotypes, p=0.003, Table 3). Promoter and intron 2 polymorphisms in the study population of the present invention were in partial linkage disequilibrium (r²=0.04; D′=0.40), which indicates that the promoter and intron 2 regions of the 5-HTT gene may play independent roles in determining drug response. The NET G1287A polymorphism showed no association with response to SSRI drugs (p=0.71 by multiple logistic regression; OR=0.84; 95% CI, 0.34-2.09).

Transporter Gene Polymorphisms and Differential Drug Response

Although it was not a primary focus of this study, the present inventors compared the response rates to NRI and SSRI by genotype (Table 3). As a result, only one strong association was detected: patients carrying the GG polymorphism of NET G1287A had a higher rate of response to NRI treatment (83.3% (35/42)) than to SSRI treatment (58.7% (44/75)). This difference is statistically significant (p=0.006 by chi-square; OR=3.52; 95% CI (1.39-8.95)).

Combinations of Transporter Polymorphisms and Response

The present inventors examined combinations of two significant polymorphisms for their association with response to each class of drug. Patients carrying unfavorable genotypes of both polymorphisms (NET A of G1287A polymorphism+promoter l of 5-HTTLPR) showed the lowest response rate among four genotype combination groups (Table 4) on NRI response. The response rate of this group (21.7%) was significantly lower than those of the other three genotype groups (62.5%, 75.0%, and 88.5%; p=0.02, p=0.008, and p<0.001 for ‘NET A+promoter ss’, ‘NET GG+promoter l’, and ‘NET GG+promoter ss’, respectively). In the SSRI group, patients carrying favorable genotypes of both 5-HTT intron 2 and promoter polymorphisms (intron2 ll+promoter ss) had the highest response rate to SSRIs. The response rate of this genotype group to SSRIs (77.4%) was higher than those of the other 3 genotype groups (54.3%, 25.0%, and 0%; p=0.06, p=0.01, and p<0.001 for ‘intron2 ll+promoter l’, ‘intron2 s+promoter ss’, and ‘intron2 s+promoter l’, respectively).

TABLE 4
Response rates to combinations of monoamine
transporter polymorphisms

| | | | | |
|---|---|---|---|---|
| | 5-HTT | | Response rate | |
| NET G1287A | promoter | 5-HHT intron 2 | (%) | P* |

NRI-treated group

| | | | | |
|---|---|---|---|---|
| GG | ss | any genotype | 23/26 (88.5) | <.001 |
| GG | l carrier | any genotype | 12/16 (75.0) | 0.008 |
| A carrier | ss | any genotype | 15.24 (62.5) | 0.02 |
| A carrier | l carrier | any genotype | 5/23 (21.7) | 0.02 |

SSRI-treated group

| | | | | |
|---|---|---|---|---|
| any genotype | ss | ll | 48/62 (77.4) | 0.06 |
| any genotype | l carrier | ll | 19/35 (54.3) | 0.06 |
| any genotype | ss | s allele carriers | 2/8 (25.0) | 0.01 |
| any genotype | l carrier | s allele carriers | 0/14 | <.001 |

Abbreviations: NET, norepinephrine transporter;
5-HTT, serotonin transporter.
*Fisher's exact test

As described above, the present invention provides a method enabling an antidepressant agent having high treatment success rate to be selected based on the combination of monoamine transporter gene polymorphisms.

### REFERENCES

1. Smeraldi E, Zanardi R, Benedetti F, Di Bella D, Perez J, Catalano M. Polymorphism within the promoter of the serotonin transporter gene and antidepressant efficacy of fluvoxamine. Mol Psychiatry. 1998; 3:508-511.
2. Pollock B G, Ferrell R E, Mulsant B H, et al. Allelic variation in the serotonin transporter promoter affects onset of paroxetine treatment response in late-life depression. Neuropsychopharmacology. v2000; 23:587-590.
3. Zanardi R, Serretti A, Rossini D, et al. Factors affecting fluvoxamine antidepressant activity: influence of pindolol and 5-HTTLPR in delusional and nondelusional depression. Biol Psychiatry. 2001; 50:323-330.
4. Rausch J L, Johnson M E, Fei Y J, et al. Initial conditions of serotonin transporter kinetics and genotype: influence on SSRI treatment trial outcome. Biol Psychiatry. 2002; 51:723-732.
5. Arias B, Catalan R, Gasto C, Gutierrez B, Fananas L. 5-HTTLPR polymorphism of the serotonin transporter gene predicts non-remission in major depression patients treated with citalopram in a 12-weeks follow up study. J Clin Psychopharmacol. 2003; 23:563-567.
6. Kim D K, Lim S W, Lee S, et al. Serotonin transporter gene polymorphism and antidepressant response. Neuroreport. 2000; 11:215-219.
7. Yoshida K, Ito K, Sato K, et al. Influence of the serotonin transporter gene-linked polymorphic region on the antidepressant response to fluvoxamine in Japanese depressed patients. Prog Neuropsychopharmacol Biol Psychiatry. 2002; 26:383-386
8. Murphy G M, Jr., Hollander S B, Rodrigues H E, Kremer C, Schatzberg A F. Effects of the serotonin transporter gene promoter polymorphism on mirtazapine and paroxetine efficacy and adverse events in geriatric major depression. Arch Gen Psychiatry. 2004; 61:1163-1169.
9. Perlis R H, Mischoulon D, Smoller J W, et al. Serotonin transporter polymorphisms and adverse effects with fluoxetine treatment. Biol Psychiatry. 2003; 54:879-883.
10. Durham L K, Webb S M, Milos P M, Clary C M, Seymour A B. The serotonin transporter polymorphism, 5HTTLPR, is associated with a faster response time to sertraline in an elderly population with major depressive disorder. Psychopharmacol. 2004; 174:525-529.
11. Minov C, Baghai T C, Schule C, et al. Serotonin-2A-receptor and -transporter polymorphisms: lack of association in patients with major depression. Neurosci Lett. 2001; 303:119-122.
12. Yoshida K, Takahashi H, Higuchi H, et al. Prediction of antidepressant response to milnacipran by norepinephrine transporter gene polymorphisms. Am J Psychiatry. 2004; 161:1575-1580.
13. Jonsson E G, Nothen M M, Gustavsson J P, et al. Polymorphisms in the dopamine, serotonin, and norepinephrine transporter genes and their relationships to monoamine metabolite concentrations in CSF of healthy volunteers. Psychiatry Res. 1998; 79:1-9.
14. Stober G, Hebebrand J, Cichon S, et al. Tourette syndrome and the norepinephrine transporter gene: results of a systematic mutation screening. Am J Med Genet. 1999; 88:158-163.
15. Runkel F, Bruss M, Nothen M M, Stober G, Propping P, Bonisch H. Pharmacological properties of naturally occurring variants of the human norepinephrine transporter. Pharmacogenetics. 2000; 10:397-405.
16. Lingjaerde O, Ahlfors U G, Bech P, Dencker S J, Elgen K. The UKU side effect rating scale. A new comprehensive rating scale for psychotropic drugs and a cross-sectional study of side effects in neuroleptic-treated patients. Acta Psychiatr Scand Suppl. 1987; 334:1-100.
17. First M B, Spitzer R L, Gibbon M, Williams J B W. Structured Clinical Interview for DSM-IV Axis I Disorders SCID I: Clinician Version, Administration Booklet. Washington, D.C.: American Psychiatric Press; 1997.
18. Hong Ng C, Norman T R, Naing K O, et al. A comparative study of sertraline dosages, plasma concentrations, efficacy and adverse reactions in Chinese versus Caucasian patients. Int Clin Psychopharmacol. 200621:87-92.
19. Keller MB. Past, present, and future directions for defining optimal treatment outcome in depression: remission and beyond. JAMA. 2003; 289:3152-3160.
20. Orsulak P J, Liu P K, Akers L C. Antidepressant drugs. In: Shaw L, Ed. The Clinical Toxicology Laboratory. USA, AACC Press; 2001
21. Tournel G, Houdret N, Hedouin V, Deveau M, Gosset D, Lhermitte M. High-performance liquid chromatographic method to screen and quantitate seven selective serotonin reuptake inhibitors in human serum. J Chromatogr B Biomed Sci Appl. 2001; 761:147-158.
22. Kollroser M, Schober C. Simultaneous determination of seven tricyclic antidepressant drugs in human plasma by direct-injection HPLC-APCI-MS-MS with an ion trap detector. Ther Drug Monit. 2002; 24:537-544.
23. Abecasis G R, Cookson W O. GOLD-graphical overview of linkage disequilibrium. Bioinformatics. 2000; 16:182-183.
24. Delgado P L, Price L H, Miller H L, et al. Rapid serotonin depletion as a provocative challenge test for patients with major depression: relevance to antidepressant action and the neurobiology of depression. Psychopharmacol Bull. 1991; 27:321-330.
25. Delgado P L, Miller H L, Salomon R M, et al. Monoamines and the mechanism of antidepressant action: effects of catecholamine depletion on mood of patients treated with antidepressants. Psychopharmacol Bull. 1993; 29:389-396.
26. Stober G, Nothen M M, Porzgen P, et al. Systematic search for variation in the human norepinephrine transporter gene: identification of five naturally occurring missense mutations and study of association with major psychiatric disorders. Am J Med Genet. 1996; 67:523-532.
27. Owen D, Du L, Bakish D, Lapierre Y D, Hrdina P D. Norepinephrine transporter gene polymorphism is not associated with susceptibility to major depression. Psychiatry Res. 1999; 87:1-5.
28. Leszczynska-Rodziewicz A, Czerski P M, Kapelski P, et al. A polymorphism of the norepinephrine transporter gene in bipolar disorder and schizophrenia: lack of association. Neuropsychobiology. 2002; 45:182-185.
29. Samochowiec J, Kucharska-Mazur J, Kaminski R, et al. Norepinephrine transporter gene polymorphism is not associated with susceptibility to alcohol dependence. Psychiatry Res. 2002; 111:229-233.
30. Sand P G, Mori T, Godau C, et al. Norepinephrine transporter gene (NET) variants in patients with panic disorder. Neurosci Lett. 2002; 333:41-44.
31. Jonsson E G, Nothen M M, Gustavsson J P, et al. Polymorphisms in the dopamine, serotonin, and norepinephrine transporter genes and their relationships to monoamine metabolite concentrations in CSF of healthy volunteers. Psychiatry Res. 1998; 79:1-9.
32. Yang L, Wang Y F, Li J, Faraone S V. Association of norepinephrine transporter gene with methylphenidate response. J Am Acad Child Adolesc Psychiatry. 2004; 43:1154-1158.
33. Tsapakis E M, Checkley S, Kerwin R W, Aitchison K J. Association between the serotonin transporter linked polymorphic region gene and response to tricyclic antidepressants. Eur Neuropsychopharmacol. 2005; 15:S26-S27.
34. Koch S, Perry K W, Nelson D L, Conway R G, Threlkeld P G, Bymaster F P. R-fluoxetine increases extracellular DA, NE, as well as 5-HT in rat prefrontal cortex and hypothalamus: an in vivo microdialysis and receptor binding study. Neuropsychopharmacology. 2002; 27:949-959.
35. Adell A, Celada P, Abellan M T, Artigas F. Origin and functional role of the extracellular serotonin in the midbrain raphe nuclei. Brain Res Brain Res Rev. 2002; 39:154-180.
36. Nystrom C, Hallstrom T. Double-blind comparison between a serotonin and a noradrenaline reuptake blocker in the treatment of depressed outpatients. Clinical aspects. Acta Psychiatr Scand. 1985; 72:6-15.
37. Benedetti F, Colombo C, Serretti A, et al. Antidepressant effects of light therapy combined with sleep deprivation are influenced by a functional polymorphism within the promoter of the serotonin transporter gene. Biol Psychiatry. 2003; 54:687-692.
38. Nakamura M, Ueno S, Sano A, Tanabe H. The human serotonin transporter gene linked polymorphism (5-HTTLPR) shows ten novel allelic variants. Mol Psychiatry. 2000; 5:32-38.
39. Gelernter J, Kranzler H, Cubells J F. Serotonin transporter protein (SCL6A4) allele and haplotype frequencies and linkage disequilibria in African- and European-American and Japanese populations and in alcohol-dependent subjects. Hum Genet. 1997; 101:243-246.
40. Bellivier F, Henry C, Szoke A, et al. Serotonin transporter gene polymorphisms in patients with unipolar or bipolar depression. Neurosci Lett. 1998; 255:143-146.
41. Fredman S J, Fava M, Kienke A S, White C N, Nierenberg A A, Rosenbaum J F. Partial response, nonresponse, and relapse with selective serotonin reuptake inhibitors in major depression: a survey of current “next-step” practice. J Clin Psychiatry. 2000; 61:403-408.
42. Anderson IM. Selective serotonin reuptake inhibitors versus tricyclic antidepressants: a meta-analysis of efficacy and tolerability. J Affect Disord. 2000; 58:19-36.
43. Hachisu M, Ichimaru Y. Pharmacological and clinical aspects of fluvoxamine (Depromel), the first selective serotonin reuptake inhibitor approved for clinical use employed in Japan. Nippon Yakurigakua Zasshi. 2000; 115:271-279.
44. Srisurapanont M. Response and discontinuation rates of newer antidepressants: a meta-analysis of randomized controlled trials in treating depression. J Med Assoc Thai. 1998; 81:387-392.
45. Serreti A, Artioli P, Quartesan R. Pharmacogenetics in the treatment of depression: pharmacodynamic studies. Pharmacogenet Genomics. 200515:61-67.
46. Heils A, Teufel A, Petri S, et al. Allelic variation of human serotonin transporter gene expression. J Neurochem. 1996; 66:2621-2624.
47. Lesch K P, Bengel D, Heils A, et al. Association of anxiety-related traits with a polymorphism in the serotonin transporter gene regulatory region. Science. 29 1996; 274:1527-1531.
48. Heinz A, Jones D W, Mazzanti C, et al. A relationship between serotonin transporter genotype and in vivo protein expression and alcohol neurotoxicity. Biol Psychiatry. 200047:643-649.
49. Ebmeier K P, Donaghey C, Steele J D. Recent developments and current controversies in depression. Lancet. 2006; 367:153-167.
50. McGue M, Christensen K. Genetic and environmental contributions to depression symptomatology: evidence from Danish twins 75 years of age and older. J Abnorm Psychol. 1997; 106:439-448.
51. Golimbet V E, Alfimova M V, Shcherbatykh T V, Rogaev E I. Allele polymorphism of the serotonin transporter gene and clinical heterogeneity of depressive disorders. Genetika. 2002; 38:671-677.
52. Steffens D C, Svenson I, Marchuk D A, et al. Allelic differences in the serotonin transporter-linked polymorphic region in geriatric depression. Am J Geriatr Psychiatry. March-April 2002; 10:185-191.
53. Reynolds C F 3rd, Dew M A, Frank E, et al. Effects of age at onset of first lifetime episode of recurrent major depression on treatment response and illness course in elderly patients. Am J Psychiatry. 1998; 155:795-799.
54. Reynolds C F, 3rd, Frank E, Kupfer D J, et al. Treatment outcome in recurrent major depression: a post hoc comparison of elderly (“young old”) and midlife patients. Am J Psychiatry. 1996; 153:1288-1292.
55. Driscoll H C, Basinski J, Mulsant B H, et al. Late-onset major depression: clinical and treatment-response variability. Int J Geriatr Psychiatry. 2005; 20:661-667 Peacock M, Turner C H, Econs M J, Foroud T 2002 Genetics of osteoporosis. Endocr Rev 23:303-326.

What is claimed is:

1. A marker of norepinephrine transporter (NET) gene polymorphism for predicting antidepressant treatment response, wherein the gene polymorphism is G1287A and is associated with the treatment effect of a noradrenaline reuptake inhibitor (NRI) antidepressant.

2. A method for predicting treatment response to an NRI antidepressant or screening a drug, the method comprising the steps of:
(i) collecting a DNA sample from a subject and detecting the gene of claim 1 in the DNA sample; and
(ii) examining polymorphisms in the gene detected in the step (i).

3. A method for predicting antidepressant treatment response, comprising the steps of:
(i) collecting a DNA sample from a subject and detecting monoamine transporter genes, a noradrenaline transporter gene and a serotonin transporter gene, in the collected DNA sample; and
(ii) examining polymorphism G1287A in the noradrenaline transporter gene, promoter region polymorphism (5-HTTLPR) in the serotonin transporter gene (5-HTT, and intron 2 polymorphism in the 5-HTT gene.

4. A method for selecting a noradrenaline reuptake inhibitor antidepressant or a selective serotonin reuptake inhibitor antidepressant, the method comprising the steps of:
(i) collecting a DNA sample from a subject and detecting monoamine transporter genes, a noradrenaline transporter gene and a serotonin transporter gene, in the collected DNA sample; and
examining polymorphism G1287A in the noradrenaline transporter gene, promoter region polymorphism (5-HTTLPR) in the serotonin transporter gene (5-HTT), and intron 2 polymorphism in the 5-HTT gene.

5. The method of claim 4, wherein, if the genotype of the noradrenaline transporter gene polymorphism G1287A is GG, and the polymorphism of the 5-HTT gene promoter is ss, the noradrenaline reuptake inhibitor antidepressant is selected.

6. The method of claim 4, wherein, if the polymorphism of the 5-HTT gene intron 2 is ll, and the polymorphism of the 5-HTT gene promoter is ss, the selective serotonin reuptake inhibitor antidepressant is selected.

7. A kit for predicting antidepressant treatment response, comprising primers for sequence variation screening of noradrenaline transporter gene polymorphism G1287A, genotyping primers and a probe.

8. The kit of claim 7, further comprising primers for sequence variation screening of serotonin transporter gene (5-HTT) promoter region polymorphism (5-HTTLPR), primers for sequence variation screening of 5-HTT gene intron 2 polymorphism, genotyping primers and a probe.

9. The kit of claim 7, wherein the primers for sequence variation screening of noradrenaline transporter gene polymorphism G1287A have base sequences of SEQ ID NO: 1 and SEQ ID NO: 2.

10. The kit of claim 8, wherein the primers for sequence variation screening of noradrenaline transporter gene polymorphism G1287A have base sequences of SEQ ID NO: 1 and SEQ ID NO: 2, the primers for sequence variation screening of serotonin transporter gene (5-HTT) promoter region polymorphism (5-HTTLPR) have base sequences of SEQ ID NO: 3 and SEQ ID NO: 4, and the primers for sequence variation screening of 5-HTT gene intron 2 polymorphism have base sequences of SEQ ID NO: 5 and SEQ ID NO: 6.


SEQUENCE LISTING (9 sequences)

SEQ ID NO: 1 - DNA, 20 bp, Artificial
  Synthesized
tccagggagaccctaattcc

SEQ ID NO: 2 - DNA, 21 bp, Artificial
  Synthesized
ttgactttattgaaatgcggc

SEQ ID NO: 3 - DNA, 21 bp, Artificial
  Synthesized
gtcagtatcacaggctgcgag

SEQ ID NO: 4 - DNA, 22 bp, Artificial
  Synthesized
tgttcctagtcttacgccagtg

SEQ ID NO: 5 - DNA, 21 bp, Artificial
  Synthesized
ggcgttgccgctctgaattgc

SEQ ID NO: 6 - DNA, 24 bp, Artificial
  Synthesized
gagggactgagctggacaacccac

SEQ ID NO: 7 - DNA, 430 bp, Homo sapiens
tttctcgagagaggcaaggcagcctacatgagtcctgggctgcaggaggctctaggaaccctggggcctgagactgaggtccagggagaccctaattcctgcaccccacccctcctggttccctccagatgggaggcatggaggctgtcatcacgggcctggcagatgacttccaggtcctgaagcgacaccggaaactcttcacatttggcgtcaccttcagcactttccttctcgccctgttctgcataaccaaggtgagtaggggctgggctctgggtcacctgggggcctctgaggccgcatttcaataaagtcaaacattcctagccttagaactgggctgagctcagggagaacaatgcaggatccagcatcctcaattcagcggcctgacccactagggttaggcccagtagtcttcttccat

SEQ ID NO: 8 - DNA, 660 bp, Homo sapiens
ggcgctgcccctgggggtgaaattcccaagcttgttggggattctcccgcctggcgttgccgctctgaatgccagcacctaacccctaatgtccctactgcagccctcccagcatcccccctgcaacctcccagcaactccctgtacccctcctaggatcgctcctgcatcccccattatcccccccttcacccctcgcggcatcccccctgcacccccagcatcccccctgcagcccccccagcatctcccctgcacccccagcatcccccctgcagcccttccagcatccccctgcacctctcccaggatctcccctgcaacccccattatcccccctgcacccctcgcagtatcccccctgcaccccccagcatccccccatgcacccccggcatcccccctgcacccctccagcattctccttgcaccctaccagtattcccccgcatcccggcctccaagcctcccgcccaccttgcggtccccgccctggcgtctaggtggcaccagaatcccgcgcggactccacccgctgggagctgccctcgcttgcccgtggttgtccagctcagtccctctagacgctcagcccaaccggccgcacagttttcaggggtcagttcctccaagtacaaggggcggtggcttctctggagc

SEQ ID NO: 9 - DNA, 598 bp, Homo sapiens
gggagacctggggcaagaaggtggatttccttctctcagtgattggctatgctgtggacctgggcaatgtctggcgcttcccctacatatgttaccagaatggagggggtcagtatcacaggctgcgagtagaggctgtgacccagggtgggctgtgacccggagtgggctgtgacccggggtgggctgtgacccgggtgggctgcgacctggggtgggctgtgacctgggatgggctgtgacccgggtgggctgtgacctggggtgggctgtgacccgggtgggctgtgacctggggtgggctgtgacccgggtgggctgtgacctgggatgggctgtaggtcctcttgagaggccagaagacagattatgtcttttcagtcttcactggcgtaagactaggaacatgatgacttagactttcgagctggtagaaaagtcaaatctcgccaggcgtggtggctcacacctgtaatcccagcactttgggaggctgaggcaggtggatcacctgaggtcaggagttcaagaccagcctgaccaacatggcaaaaccccgtctccactaaaaatataaaaattagctgggtatggtggt